(19) United States
(12) Patent Application Publication
(10) Pub. No.: US 2013/0090690 A1
(43) Pub. Date: Apr. 11, 2013

(54) Dynamic Rod Assembly

(71) Applicant: David A. Walsh, Reading, MA (US)

(72) Inventor: David A. Walsh, Reading, MA (US)

(21) Appl. No.: 13/611,700

(22) Filed: Sep. 12, 2012

Related U.S. Application Data
(60) Provisional application No. 61/543,958, filed on Oct. 6, 2011.

(51) Int. Cl. A61B 17/70

(52) U.S. Cl. 606/257; 606/260

(57) ABSTRACT

A dynamic rod assembly, such as that used for spinal stabilization, made of a number of interlocking segments whereby a limited amount of relative motion is permitted between each pair of adjacent segments. The dynamic rod assembly may also incorporate a separate central element that extends at least partially through a central channel within the interlocking segments to prevent the interlocking segments from disengaging while adding to the desired bending properties of the dynamic rod assembly.

### FIELD OF THE INVENTION

The present invention relates generally to prostheses for treating spinal pathologies, and more specifically to dynamic stabilization rods for use with spinal fixation assemblies.

### BACKGROUND OF THE INVENTION

Various methods of spinal immobilization have been used in the treatment of spinal instability and displacement. The most common treatment for spinal stabilization is immobilization of the joint by surgical fusion, or arthrodesis. This has been known for almost a century. Early in the century, post operative external immobilization, such as through the use of splints and casts, was the favored method of spinal fixation. As surgical techniques became more sophisticated, various new methods of internal and external fixation were developed.

Internal fixation refers to therapeutic methods of stabilization that are wholly internal to the patient and include commonly known devices such as bone plates, screws, rods and pins. External fixation, in contrast, involves at least some portion of the stabilization device being located external to the patients' body. As surgical technologies and procedures became more advanced and the likelihood of infection decreased, internal fixation eventually became the favored method of immobilization since it is less restrictive on the patient.

Internal fixation of the spine may be used to treat a variety of disorders including degenerative spondylolisthesis, fracture, dislocation, scoliosis, kyphosis, spinal tumor, and failed previous fusion (pseudarthrosis). One of the main challenges associated with internal spinal fixation is securing the fixation device to the spine without damaging the spinal cord. The pedicles of a vertebra are commonly used for fixation as they generally offer an area that is strong enough to hold the fixation device in place even when the patient suffers from degenerative instability such as osteoporosis.

Current fixation devices and hardware systems generally include a fixation device, such as a screw, a rod, and a body for fixing the position of the rod with respect to the screw, which in turn fixes the rod with respect to the spine. However, because traditional metal rods are far less compliant than bone, these rigid rods can cause significantly more stress on the neighboring levels of the spine and can contribute to premature degeneration of nearby levels. The present invention provides a novel dynamic rod assembly that allows the affected spinal levels to be stabilized by limiting excessive motion while allowing a degree of mobility without transmitting excessive forces.

### BRIEF SUMMARY OF THE INVENTION

Disclosed is a rod for use with spinal fixation assemblies. The rod comprising a number of interlocking metal segments; at least one lateral channel extending there through; and at least one central element substantially filling at least one lateral channel extending through the interlocking metal segments.

Also disclosed is a dynamic rod assembly for use with spinal fixation assemblies that comprising a number of interlocking metal segments; at least one lateral channel extending there through; and at least one central element substantially filling at least one lateral channel extending through the interlocking metal segments such that the interlocking metal segments together form a dynamic rod assembly at least a portion of which is generally cylindrical.

The features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto.

Features that are described and/or illustrated with respect to one embodiment may be used in the same way or in a similar way in one or more other embodiments and/or in combination with or instead of the features of the other embodiments.

### BRIEF DESCRIPTION OF THE SEVERAL VIEWS OF THE DRAWINGS

FIG. 1A is a perspective view of a dynamic rod assembly;

FIG. 1B is a front view of the dynamic rod assembly of FIG. 1A;

FIG. 2A is a perspective view of a single interlocking middle segment of the dynamic rod assembly of FIG. 1A;

FIG. 2B is a perspective view of an interlocking female end segment of the dynamic rod assembly of FIG. 1A;

FIG. 2C is a perspective view of an interlocking male end segment of the dynamic rod assembly of FIG. 1A;

FIG. 2D is a perspective view of a central element of the dynamic rod assembly of FIG. 1A;

FIG. 2E is a perspective view of a threaded element of the dynamic rod assembly of FIG. 1A;

FIG. 3 is an exploded perspective view of the dynamic rod assembly of FIG. 1A;

FIG. 4A is a front view of a partial section of a dynamic rod assembly in its free state;

FIG. 4B is a front view of the partial section of a dynamic rod assembly of FIG. 4A with an applied load;

FIG. 4C is a side view of the partial section of a dynamic rod assembly of FIG. 4B with an applied load;

FIG. 5 is a front view of a partial section of a dynamic rod assembly showing different segment lengths; and

FIGS. 6A & B are front views of a partial section of a dynamic rod assembly showing alternate interlocking geometries.

### DETAILED DESCRIPTION OF THE INVENTION

The invention relates to novel spinal dynamic rod assemblies for use with spinal fixation assemblies. The dynamic rod assemblies preferably permit the affected spinal levels to be stabilized by limiting excessive motion while allowing a degree of mobility without transmitting excessive forces. This may be accomplished through a variety of designs, each of which includes a dynamic rod assembly made of a number of interlocking segments whereby a limited amount of relative motion is permitted between each pair of adjacent segments.

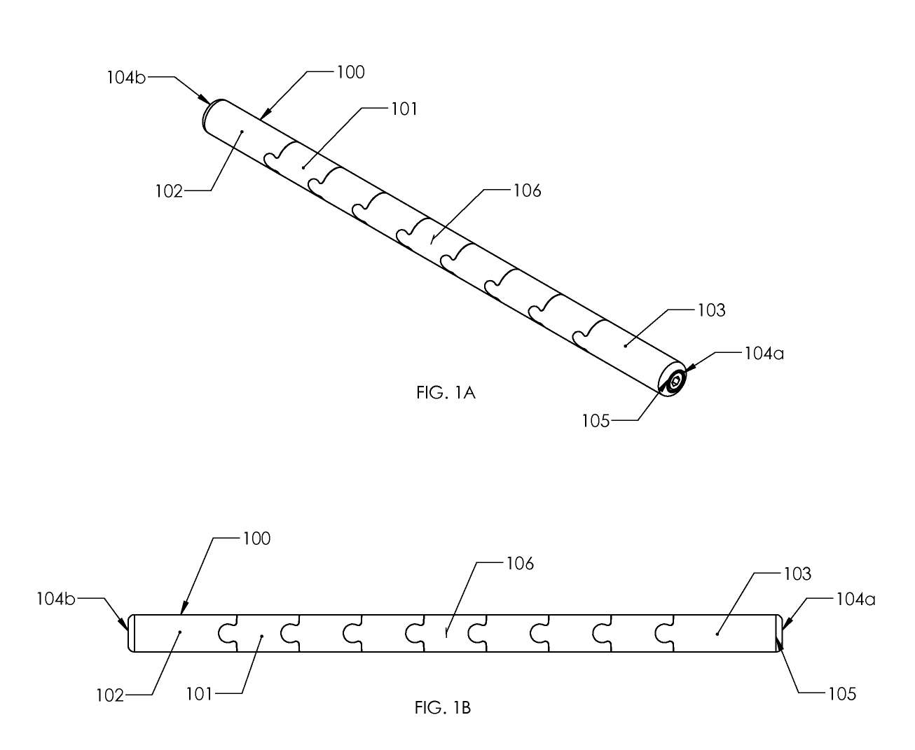

Turning initially to FIGS. 1A-B, perspective and front views of an exemplary dynamic rod assembly are illustrated. The rod 100 is made up of a number of interlocking components starting with a female end segment 102, followed by a number of middle segments 101 and lastly by a male end segment 103. A threaded element 104a is engaged within the male end segment 103 and a second threaded element 104b is engaged within the female end segment 102. The threaded elements 104a & 104b secure a captive central element 105 within the dynamic rod assembly 100 to prevent the lateral separation of adjacent segments.

The metal material 106 may include, but is not limited to, titanium, titanium alloys (e.g., titanium/aluminum/vanadium (Ti/Al/V) alloys), cobalt-chromium alloys, stainless steel, and combinations thereof, which may include mechanically compatible mixtures of the above materials, or other similar metal material(s). In the presently preferred embodiment, the metal material 106 is a Ti/AlN alloy, such as Ti/6Al/4V ELI.

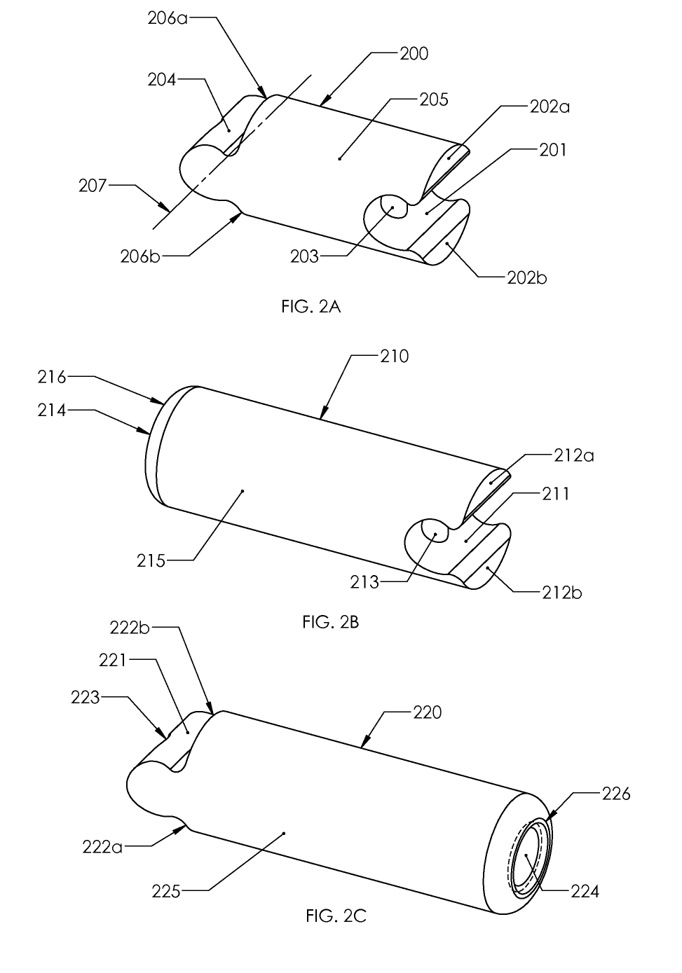

Turning next to FIGS. 2A-E each component of the dynamic rod assembly is shown in perspective view. In FIG. 2A the middle segment 200 has one female interlocking end 201 and one male interlocking end 204 with can engage the female interlocking end of a second similar segment. Although the segment shown 200 shows one male interlocking end 204 and one female interlocking end 201, those skilled in the art can appreciate that a single segment with two male interlocking ends or two female interlocking ends would function in a similar fashion.

In the preferred embodiment, the male interlock end 204 is designed to pivot around its central axis 207 when it is engaged within a female interlocking end 201. The range of motion of the pivoting is limited by coincidence of the surfaces on the male end 206a & 206b and the surfaces on the female end 202a & 202b.

The middle segment 200 of FIG. 2A has a generally cylindrical internal surface 203 along the same axis as the generally cylindrical outer surface 205 to allow engagement with a central element (shown in FIG. 2D).

Turning to FIG. 2B, the female end segment 210 has similar interlocking features 211 and similar motion limiting features 212a & 212b as the middle segment 200 of FIG 2A. Moreover, the female end segment also has a generally cylindrical internal surface 213 along the same axis as the generally cylindrical outer surface 215 to allow engagement with a central element (shown in FIG. 2D).

In this preferred embodiment, the female end segment 210 and the male end segment 220 each has a threaded internal surface (214 & 224 respectively) along its axis (213 & 223 respectively) in the end opposite that which has the interlocking feature. This threaded internal surfaces 214 & 224 allow for the engagement of a threaded element as shown in FIG. 2E. The purpose of the threaded element is to hold the central element shown in FIG. 2D captive within the dynamic rod assembly 100. However, those skilled in the art can appreciate that other means of holding the central element captive are possible such as a blind hole, welding, snap fit, or other means not herein defined.

Turning to FIG. 2C, the male end segment 220 has similar interlocking features 221 and similar motion limiting features 222a & 222b as the middle segment 200 of FIG. 2A. Moreover, the male end segment also has a generally cylindrical internal surface 223 along the same axis as the generally cylindrical outer surface 225 to allow engagement with a central element (shown in FIG. 2D).

Turning to FIG. 2D the central element 230 has a generally cylindrical outer surface 231 to engage within the generally cylindrical internal surfaces 203, 213, & 223 of the middle segment 200, the female end segment 210 and the male end segment 220 respectively.

The overall length of the central element 230 should be such that when the dynamic rod 100 is fully assembled, the surfaces 232a & 232b each are coincident with each surface 242 of the two thread elements 240 shown in FIG. 2E that are included within the dynamic rod assembly 100.

Turning to FIG. 2E the threaded element is configured in a way such that the threaded surface 241 can be engaged with the threaded internal surfaces 214 & 224 of the female end segment 210 and the male end segment 220 respectively. Furthermore, the overall length of the threaded element 240 should be configured in conjunction with the overall length of the central element 230 such that when the dynamic rod 100 is assembled the surface 243 of one threaded element 240 is generally coincident with the surface 216 of the female end segment 210 and the surface 242 of the same threaded element 240 is generally coincident with the surface 232a of the central element 230. Additionally, the surface 243 of a second threaded element 240 is generally coincident with the surface 226 of the male end segment 220 and the surface 242 of the same threaded element 240 is generally coincident with the surface 232b of the central element 230.

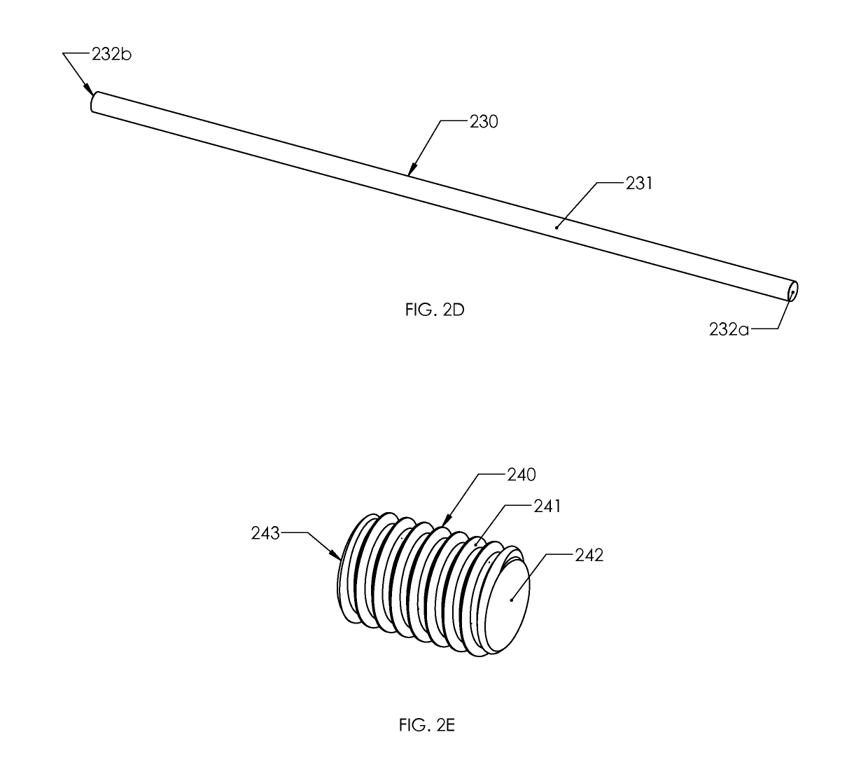

Turning now to FIG. 3, the exploded view of the dynamic rod assembly shows the assembly sequence of the preferred embodiment. Each assembly requires the following components: 1 female end segment 210; 1 male end segment 220; 1 central element 230; 2 threaded elements 240a & 240b; and multiple middle segments 220. The number of middle segments is determined by the overall length of the assembly 100 required for a specific spinal surgical procedure.

To assembly the dynamic rod 100 the male end 204 of the first middle segment 200a is slid in laterally to the interlocking feature 211 of the female end segment 210, following that the male end 204 of each subsequent middle segment 200b is slid in laterally to the female interlocking feature 201 of the previous middle segment 200. This process continues for all the middle segments 200. Then, the male interlocking feature 221 of the male end segment 220 is slid in laterally to the female end 201 of the final middle segment 200. Following this, the central element 230 is inserted into the generally cylindrical internal surfaces 213, 203, & 223 of the female end segment 210, the middle segments 200 and the male end segment 220 respectively. Finally, the two threaded elements 240a & 240b are engaged with the internal threaded surfaces 214 & 224 of the female end segment 210 and the male end segment 220 respectively.

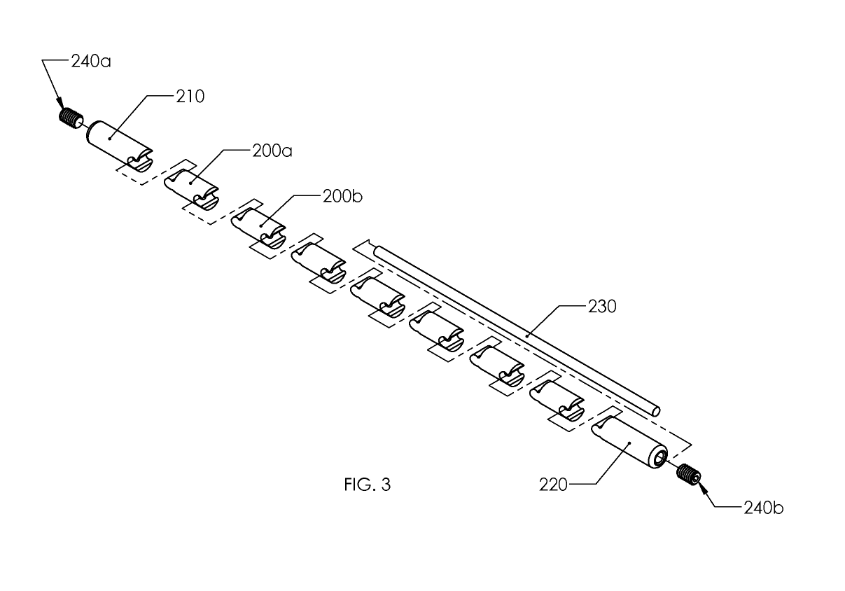

Turning now to FIGS. 4A-C the dynamic bending characteristics of the rod assembly 100 are illustrated. These figures are meant to show the bending and motion between two adjacent segments. The overall bending properties of the dynamic rod assembly 100 will be an accumulation of the bending properties between all adjacent segments within a complete assembly.

FIG. 4A shows a representative sample of middle segments 401a, 401b, & 401c in their free state without an applied load. Each male interlocking feature is concentric to its respective female interlocking feature (see 404a & 404b). In this state there is a gap at the top of each joint 402a & 402b and at the bottom of each joint 403a & 403b. When the gaps 402 & 403 are present, the bending properties of the rod result from the bending properties of the central element.

FIG. 4B shows the same representative sample of middle segments 411a, 411b, & 411c but with an applied load. The upward force 415 is applied to the center of segment 411b and is perfectly balanced with the downward force 416a & 416b applied to the ends of segments 411c & 411a respectively. The balanced forces result in an equilibrium and a steady state condition. The relative motion between the adjacent segments is limited by the elimination of the gap on the bottom side of the assembly 413a & 413b. A corresponding increase in the gap on the top side of the assembly 412a & 412b also occurs.

Once a sufficient load is applied that the gaps 413a & 413b are closed then the bending properties of the dynamic rod assembly are determined by both the interlocking segment and the central element. It is in this way that the dynamic rod assembly exhibits non-linear bending characteristics. Thus the dynamic rod assembly allows limited initial range of motion but greatly restricts excessive range of motion of the spine.

Furthermore, FIG. 4C shows the same representative sample of middle segments 411a, 411b, & 411c with an applied load but from a side view. It can be seen that due to the geometry of the interlocking features, there is no relative motion between adjacent segments in this plane. It is this feature that results in different bending properties from side to side than from front to back.

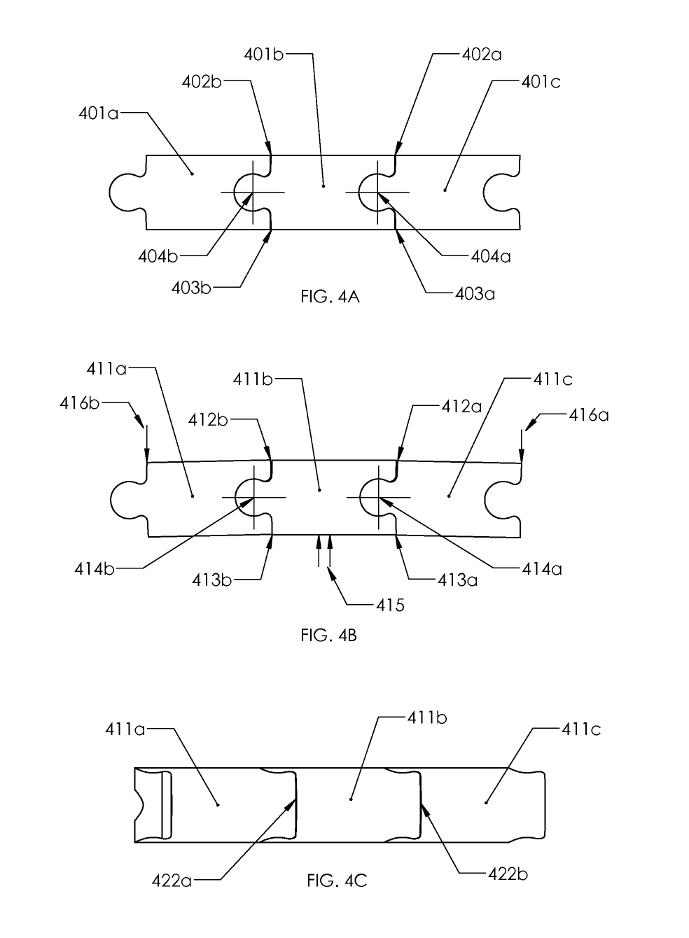

Turning now FIG. 5, the non-linear bending properties can be further enhanced by varying the length of the middle segments 501, 502, & 503 when assembling the rod. For a given length dynamic rod assembly, the sum of the gap distances 412a & 412b determines the initial bending properties of the rod. If the dynamic rod assembly is made up of fewer longer middle segments such as 503 the sum of the gap distances will be less than a dynamic rod assembly made up of shorter middle segments such as 501.

Longer middle segments, such as 503, will result in an increased resistance to bending and a decreased initial range of motion, while shorter middle segments, such as 501, will have the opposite effect, decreased resistance to bending and an increased initial range of motion. One embodiment could include middle segments of various sizes within the same rod to provide bending properties specific to a surgical need.

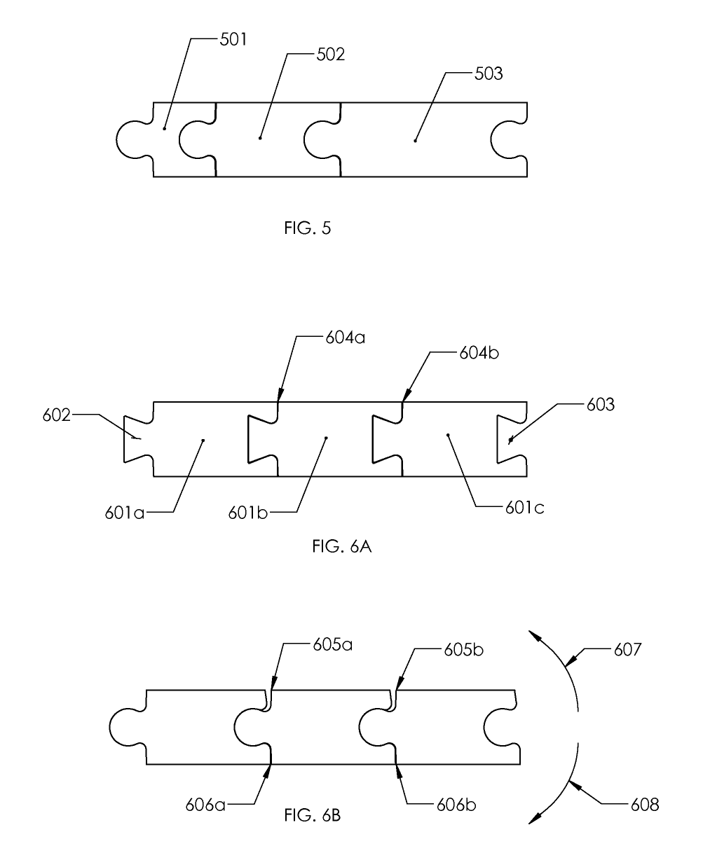

FIG. 6a shows an alternative embodiment of the invention, in which the geometry of the interlocking mechanism has a more triangular shape for both the male and female ends 602 and 603 than circular (see FIGS. 404a & 404b). With alternative geometries, gap distances 604a & 604b are still present to provide non-linear bending characteristics as in the preferred embodiment. As in the preferred embodiment, the middle segments 601a, 601b and 601c can vary in length to provide various bending properties.

A second alternative embodiment is shown in FIG. 6b. In this figure, the gaps 605a & 605b are greater than the gaps 606a & 606b, resulting in less resistance to bending in direction 607 as compared to direction 608. As in other described embodiments, these non-symmetrical segments shown in FIG. 6b can vary in length within the same dynamic rod assembly and can be combined with segments such as those shown in FIG. 5 and FIG. 4a to provide bending properties specific to a surgical need.

While the present invention has been described in association with exemplary embodiments, the described embodiments are to be considered in all respects as illustrative and not restrictive. Such other features, aspects, variations, modifications, and substitution of equivalents may be made without departing from the spirit and scope of this invention, which is intended to be limited only by the scope of the following claims. Also, it will be appreciated that features and parts illustrated in one embodiment may be used, or may be applicable, in the same or in a similar way in other embodiments.

Although the invention has been shown and described with respect to certain embodiments, it is obvious that certain equivalents and modifications may be apparent to those skilled in the art upon the reading and understanding of the specification. The present invention includes all such equivalents and modifications, and is limited only by the scope of the following claims.

1. A dynamic rod assembly for use with spinal fixation assemblies comprising a number of interlocking metal segments whereby a limited amount of relative motion is permitted between each pair of adjacent segments; at least one lateral channel extending there through; and at least one central element substantially filling at least one lateral channel extending through the interlocking metal segments.

2. The dynamic rod assembly of claim 1 wherein at least part of at least one interlocking segment comprises at least part of at least one central element

3. The dynamic rod assembly of claim 1 wherein at least part of the rod has a different bending moment from side to side than front to back.

4. The dynamic rod assembly of claim 1 wherein the central element prevents the interlocking segments from disengaging.

5. The dynamic rod assembly of claim 1 wherein the central element resembles a cylinder.

6. The dynamic rod assembly of claim 5 wherein the central element is captive within a lateral channel extending through interlocking metal segments by means of at least one threaded element.

7. The dynamic rod assembly of claim 6 wherein at least one threaded element is engaged laterally.

8. The dynamic rod assembly of claim 1 wherein the first and last interlocking metal segments do not have an interlocking feature on one end.

9. The dynamic rod assembly of claim 1 wherein each interlocking metal segment has a male interlocking feature on one end and a female interlocking feature on the opposite end with which to interlockingly engage with adjacent segments.

10. The dynamic rod assembly of claim 1 wherein the length of the metal segments is not uniform.

11. A dynamic rod assembly for use with spinal fixation assemblies comprising a number of interlocking metal segments whereby a limited amount of relative motion is permitted between each pair of adjacent segments; at least one lateral channel extending there through; and at least one central element substantially filling at least one lateral channel extending through the interlocking metal segments such that the interlocking metal segments together form a dynamic rod assembly at least a portion of which is generally cylindrical.

12. The dynamic rod assembly of claim 11 wherein at least part of at least one interlocking segment comprises at least part of at least one central element.

13. The dynamic rod assembly of claim 11 wherein at least part of the rod has a different bending moment from side to side than from front to back.

14. The dynamic rod assembly of claim 11 wherein at least part of the rod is more resistant to bending in a first direction than in a second direction opposite the first direction.

15. The rod of claim 11 wherein the central element resembles a cylinder.

16. The dynamic rod assembly of claim 15 wherein the central element is captive within a lateral channel extending through interlocking metal segments by means of at least one threaded element.

17. The dynamic rod assembly of claim 16 wherein at least one threaded element is engaged laterally.

18. The dynamic rod assembly of claim 11 wherein the first and last interlocking metal segments do not have an interlocking feature on one end.

19. The dynamic rod assembly of claim 11 wherein each interlocking metal segment has a male interlocking feature on one end and a female interlocking feature on the opposite end with which to interlockingly engage with adjacent segments.

20. The rod of claim 11 wherein the length of the metal segments is not uniform.
